(19) United States
(12) Patent Application Publication
(10) Pub. No.: US 2009/0156462 A1
(43) Pub. Date: Jun. 18, 2009

(54) USE OF FIBROUS TISSUE INDUCING PROTEINS FOR HERNIA REPAIR

(75) Inventors: James H. Pickar, Springfield, PA (US); Joanne M. Archambault, Arlington, MA (US); Howard Seeherman, Cambridge, MA (US)

Correspondence Address: WYETH/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 12/389,171

(22) Filed: Feb. 19, 2009

Related U.S. Application Data
(63) Continuation of application No. 11/386,749, filed on Mar. 23, 2006.
(60) Provisional application No. 60/664,933, filed on Mar. 24, 2005.

(51) Int. Cl. A61K 38/02; A61B 17/03

(52) U.S. Cl. 514/2; 606/151

(57) ABSTRACT

The present disclosure relates to hernia repair and methods for stimulating growth of fascia tissue employing compositions comprising fibrous tissue inducing proteins.

### CROSS REFERENCE TO RELATED APPLICATIONS

This application is a continuation of U.S. patent application Ser. No. 11/386,749, filed on Mar. 23, 2006, which claims the benefit of U.S. Patent Application No. 60/664,933, filed on Mar. 24, 2005, both of which are herein incorporated by reference in their entirety.

### FIELD OF THE INVENTION

This invention relates to the field of hernia repair and other methods of strengthening or repairing the fascia tissue.

### BACKGROUND OF THE INVENTION

A hernia is a fascia defect in a structure, such as, for example, the abdominal wall, through which an organ, part of an organ, a tissue, or part of a tissue may protrude. Usually it involves the weakening, bulging, or actual tearing of the fascia in a structure which normally contains an organ or tissue. There are many types of hernias. For example, when in the lower abdominal area, a hernia often involves intra-abdominal contents, such as the intestines or other tissue, which pass into or through a defect in the abdominal wall. There are at least two types of hernias that occur in the groin region, inguinal and femoral. A femoral hernia, which is more common in women than men, involves penetration of a tissue or an organ through the femoral ring. Inguinal hernia involves penetration of an organ or a tissue through the superficial inguinal ring. An indirect inguinal hernia leaves the abdominal cavity at the internal ring and passes down the inguinal canal, whereas a direct hernia protrudes through the floor of the inguinal canal in the Hesselbach's triangle. Hernias that occur in the abdominal wall at sites other than the groin are referred to as ventral hernias. Examples of ventral hernias include umbilical and incisional hernias. Other types of hernias are well characterized in surgical texts.

Known causes of hernia include obesity, pregnancy, tight clothing, sudden physical exertion, such as weight lifting, coughing, and abdominal injury. According to the National Center for Health Statistics, approximately five million Americans develop hernia each year. Inguinal hernias are more common in men, primarily because of the unsupported space left in the groin after the testicles descend into the scrotum. Whereas hernias in the femoral area, at the top of the thigh, are more common in women and commonly result from pregnancy and childbirth.

Temporary relief from the symptoms of some hernias can be obtained by the patient wearing a truss device that applies external pressure against the abdomen in the region of the hernia. This well known and long-established treatment rarely, if ever, provides more than temporary relief from pain and can result in discomfort to the patient from wearing the device. Permanent relief typically requires invasive surgery to return the offending organ or tissue, if present, to its original and correct position, followed by the repair and reinforcement of the fascia defect in the structure which normally contains the organ or tissue.

Additionally, mesh-type patches have been used to repair openings or holes formed in a structure through which interior organs or tissues may protrude. Typically, these patches are permanently implanted in a patient's body and may cause postoperative discomfort to the patient. Further, they have been reported to have a likelihood of harboring bacteria, thereby leading to infections.

Although mesh-type patches are widely used for hernia repair, recurrence is a problem frequently associated with their use. Recurrence has been attributed, at least in part, to the length of time required for hernia repair, which often is not met, for example, either because the mesh-type patches are displaced after a period of time after implantation in a patient, or they fail to remain in the body long enough for adequate repair, such as in the case of bioabsorbable meshes.

### SUMMARY OF THE INVENTION

The present invention is directed to methods of stimulating growth of the fascia tissue in a subject. Fascia is a sheet or band of fibrous connective tissue enveloping, separating, or binding together muscles, organs, and other soft structures of the body. Stimulation of growth of fascia tissue is important in, e.g., treating hernias, which often include damage to or a defect in fascia tissue. Surgical implants and compositions described herein are especially useful for the repair of fascia tissue defects, such as hernias, in the abdominal cavity, including inguinal (direct and indirect), femoral, incisional, and recurrent hernias.

Specifically, the invention provides compositions and devices for treating a fascia tissue defect and related methods that comprise fibrous tissue inducing proteins, e.g., members of the bone morphogenetic protein (BMP) family such as, e.g., BMP-12, BMP-13, or MP-52. Such compositions may further comprise a tissue adhesive, e.g., fibrin. The use of such compositions will result in faster and/or more effective repair of the fascia. A composition comprising one or more fibrous tissue inducing proteins (and optionally one or more tissue adhesives) may be delivered to the site of a fibrous tissue defect directly or by using a surgical implant, such as, e.g., a mesh. Alternatively, a composition comprising one or more fibrous tissue inducing proteins and a separate composition comprising one or more tissue adhesives may be delivered directly to the site of a fascia tissue defect or by using a surgical implant. Suitable fascia tissue defect repair implants of varying sizes and shapes can be anchored to the surrounding healthy tissue to prevent migration. Implants can also be configured to substantially occlude and conform to the walls of a fascia defect, e.g., in a hernia.

Methods of making and using the compositions and devices of the invention are also provided.

### DESCRIPTION OF THE EMBODIMENTS

Surgical implants, compositions, and methods described herein generally relate to treating defects of fascia tissue, such as, e.g., in hernia repair. More particularly, surgical implants, compositions, and methods employ fibrous tissue inducing proteins, e.g., members of the bone morphogenetic protein (BMP) family such as, e.g., BMP-12, BMP-13, or MP-52. Evidence suggests that a defect in the metabolism of collagen is involved in the pathogenesis of certain types of hernias, such as, for example, inguinal hernia in adults, leading to a weakening of the transversalis fascia tissue, which poses a problem for effective repair of hernias as well as increases the likelihood of recurrence following repair. When fascia has been traumatized, it heals with a special type of collagen fiber called type III. Thus, by way of theory and not as a limitation, it is hypothesized that the fibrous tissue inducing proteins of the inventions may contribute to correction of the collagen metabolism, thereby treating a defect of fascia.

In general, the invention provides a method of treating a defect of fascia tissue, comprising delivering a composition comprising a fibrous tissue inducing protein to the site of the fascia defect. Such compositions may further comprise a tissue adhesive, e.g., fibrin. Compositions may be delivered to the site of a hernia directly or by using an implantable device such as, e.g., a surgical implant suitable for repair of a fascia tissue defect. Surgical implants, compositions, and methods are described in detail below.

### Fibrous Tissue Inducing Proteins

Fibrous tissue inducing proteins used in the compositions, implants, and methods of the invention are selected from the family of proteins known as the transforming growth factors beta (TGF-β) superfamily. This family includes activins, inhibins, and bone morphogenetic proteins (BMPs). Certain BMPs are particularly useful in inducing fibrous tissue growth. In preferred embodiments, the fibrous tissue inducing protein is chosen from BMP-12, BMP-13 and MP-52 (also known as GDF-7, GDF-6, and GDF-5, respectively), which form a subgroup of in the BMP family. The nucleotide and protein sequences of BMP-12, BMP-13 and MP-52 are disclosed in U.S. Pat. No. 5,658,882 and their database accession numbers are shown in Table 1.

TABLE 1

| Fibrous tissue | | |
|---|---|---|
| inducing protein | Human | Mouse |
| BMP-12 | Q7Z4P5 | P43029 |
| BMP-13 | Q6KF10 | P43028 |
| MP-52 | P43026 | P43027 |

Nucleotide and protein sequences for other BMP and TGF-β family members are well known in the art.

Other candidate proteins that may be useful in repair of fascia tissue defects can also be identified using one or more assays described herein to evaluate hernia repair, for example, by measuring the tissue integration strength in the presence of a candidate protein, or by measuring collagen (especially collagen type III) section by cell in vitro or in vivo. BMP-13 and MP-52 are 86% identical to each other, and 80% identical to BMP-12, whereas they are only 57% identical to next most homologous member of the TGF-β superfamily, BMP-2 (See, e.g., FIG. 4 of U.S. Pat. No. 6,096,506). Thus, it is expected that a protein that is a least about 70% identical to any one of BMP-12, BMP-13 and MP-52 would possess the required fibrous tissue inducing activity. Accordingly, some embodiments include the use of a fibrous tissue inducing protein that is, for example, 70%, 75%, 80%, 85%, 90%, 95%, 98%, 99% identical to BMP-12, BMP-13 or MP-52. Such proteins can be engineered, for example, by mutating or deleting a number of non-conserved amino acid residues, for example, those residues that differ between the corresponding mouse and human sequences (or other species) and or those residues that differ between any two of BMP-12, BMP-13, and MP-52, when sequences are aligned. Conservative amino acid substitutions in native sequences are also contemplated. Alternatively, fragments of such homologous or modified proteins, as well as fragments of native fibrous tissue inducing proteins, that retain fibrous tissue inducing activity may be used in the methods of the invention.

Fibrous tissue inducing proteins may either be recombinantly produced or be purified from natural sources. In the preferred embodiments, the proteins are of the human origin and are recombinant. Methods for recombinant product of proteins are well known and are described, for example, in U.S. Pat. No. 5,658,882.

In some embodiments, an effective amount of a fibrous tissue inducing protein that may be used in the compositions and implants described herein is that amount which is sufficient for repairing fascia in a subject at a rate that is 10%, 20%, 30%, 50% faster or more than the corresponding repair in the absence of the fibrous tissue inducing protein and will generally depend upon the size and nature of the fascia defect being repaired and/or the surface area of the implant being employed. In other embodiments, an effective amount of a fibrous tissue inducing protein is that amount which is sufficient for stimulating fascia tissue growth at a rate that is 10%, 20%, 30%, 50% faster or more than the growth in absence of the fibrous tissue inducing protein.

Generally, the amount of protein used for repairing a fascia defect and/or for stimulating growth of fascia tissue is in a range of from 0.001 to 10 mg, 0.01 to 1 mg, or 0.1 to 0.5 mg per cubic centimeter of material required. In some instances, dosages may be deduced from the concentration of protein in the composition applied to the mesh. For example, a composition applied to the mesh may contain from 0.001 to 10 mg/ml, from 0.01 to 1.0 mg/ml, or from 0.1 to 0.5 mg/ml of one or more fibrous tissue inducing proteins. For example, if a mesh has a 1 cc volume and can absorb an equal amount of liquid, 1 ml of composition is applied to the mesh, for a soak load of 100%. Soak loads can vary from 25% to 200%, from 50% to 150%, or from 75% to 100%. Particular dosage will be determined by the clinical indication being addressed, as well as by various patient variables (e.g., weight, age, sex) and clinical presentation (e.g., extent of and/or site of the fascia defect, etc.).

### Tissue Adhesives

Tissue adhesives for use in the compositions and surgical implants of the invention include fibrin, fibrinogen, thrombin, aprotinin, and Factor VIII. Commercially available tissue adhesives include TISSEEL® (fibrinogen; Baxter Healthcare Corp., Deerfield, Ill.) and DERMABOND™ (2-octyl cyanoacrylate; Ethicon, Somerville, N.J.). These adhesives may be combined directly with a fibrous tissue inducing protein or applied to the site of a fascia defect either before, after, or at the same time as the fibrous tissue inducing protein. The adhesives may also be incorporated into a surgical implant in the same ways as described for the fibrous tissue inducing proteins. Compositions including tissue adhesives can also be used for stimulating growth of fascia tissue. In some embodiments, tissue adhesives, alone or in combination with at least one fibrous tissue inducing protein, are delivered in a composition in the form of a paste or a gel.

### Other Additives

Additives that may be useful in the compositions and surgical implants described herein, include, without limitation, pharmaceutically acceptable salts, polysaccharides, peptides, proteins, amino acids, synthetic polymers, natural polymers, and/or surfactants. Additives which help in reducing or preventing the adhesion of surrounding tissue and organs to the surgical implant are particularly useful and are referred to herein as anti-adhesion compounds. Non-limiting examples of such additives include, for example, chemically modified sodium hyaluronate and carboxymethylcellulose (modified with the activating agent 1-(3-dimethylaminopropyl)-3-ethylcarbodimide hydrochloride (EDC) and available commercially as SEPRAFILM® adhesion barrier (Genzyme Corp., Cambridge, Mass.)), hyaluronic acid, and collagen.

In some embodiments, compositions and surgical implants described herein contain an antimicrobiotic agent, such as an antibiotic. Administration of antibiotics serves to prevent infections. Examples of antibiotics that may be used include, but are not limited to, TYGACIL® (tigecycline; Wyeth, Madison, N.J.), cephalosporins such as cephazolin and cephamandol, netilmycin, penicillins such as oxacillin or mezlocillin, tetracycline, metronidazole or aminoglycosides such as gentamycin or neomycin, and rifampicin. Generally, the amount of antibiotic used is in a range of from 0.001 to 10 mg, 0.01 to 1 mg, or 0.1 to 0.5 mg per cubic centimeter of material required.

Like the adhesives, these additives may be combined directly with the fibrous tissue inducing protein, or applied to the site of a fascia defect either before, after, or at the same time as the fibrous tissue inducing protein. The additives may also be incorporated into a surgical implant, in the same ways as described for the fibrous tissue inducing proteins.

Compositions useful in the methods of the invention may be delivered directly to a site of fascia defect. They may be applied (e.g., injected) to the site, while the defect is otherwise repaired using traditional surgical techniques. The compositions may also be used in conjunction with a surgical implant that has not been treated with such a composition. Alternatively, compositions described herein may be applied to the affected area either before or after a surgical implant is put into place.

### Surgical Implants

Surgical implants for hernia repair typically include a mesh, or other means of structural support. An implant has a structure that may serve to both release the protein in a time-dependent manner and provide structural support for hernia repair. The surgical implant may comprise at least one fibrous tissue inducing protein and, optionally, at least one tissue adhesive. The surgical implant can be treated by any method, so long as the method allows the fibrous tissue inducing protein(s) to be delivered to the site of a fascia defect in a subject. For example, a mesh may be coated with a fibrous tissue inducing protein by immersing or soaking it in a solution of fibrous tissue inducing protein(s), for example, from 1 minute to 1 hour, 10 minutes to 45 minutes, or 15 minutes to 30 minutes. Coating may be also achieved by, for example, spraying the mesh with such a solution. In yet other embodiments, a mesh may be impregnated with a fibrous tissue inducing protein by the use of chemical cross-linking.

Meshes that can be employed as surgical implants include, for example, polypropylene mesh (PPM) which has been used extensively in hernia repair to provide the necessary strength and support for tissue growth for the repair of abdominal defects in hernia. Other examples include expanded polytetraflouroethylene (ePTFE), sepramesh biosurgical composite, polyethylene terephthalate (PET), and titanium. Ideal mesh properties include, without limitation, inertness, resistance to infection at the site where the mesh is implanted, molecular permeability, pliability, transparency, mechanical integrity and strength, and biocompatibility.

Implants may have a dorsal surface and a visceral surface. The dorsal surface is the portion of the implant which faces outward away from a fascia defect and the visceral surface is the portion which faces inward towards the defect. Prior to implantation, some of the implants described herein may, in an unstressed state, assume a flat or planar shape, or may assume a concave and/or convex shape on one or more surfaces.

In yet other embodiments, an implant comprises a mesh in the form of a sponge, for example, which is soaked or immersed in a composition comprising a fibrous tissue inducing protein and optionally a tissue adhesive, so that the composition fully permeates the pores of the sponge. Such a sponge can either be made from a synthetic material, such as polyvinyl alcohol, or from a bioabsorbable material, such as collagen, gelatin, keratin, laminin, fibrin, or fibronectin. Examples include HELISTAT®, HELITENE®, and VITAGUARD® (Integra Life Sciences, Plainsboro, N.J.), and ULTRAFOAM® (Davol, Inc., Cranston, R.I.). In certain instances, it is preferable to use a bioabsorbable sponge that is only temporarily present in the body of a subject. Meshes and sponges described herein may also be referred to by other terms, such as for example, a pad or a gauze, etc.

In some embodiments, implants may be sufficiently flexible to allow a surgeon to manipulate the implant to conform to the surgical site and/or ease delivery during a laparoscopic procedure. However, in some circumstances, a stiffer arrangement that limits compression and/or expansion of the implant may be preferred. In certain embodiments, an implant may be collapsible, such as by folding, rolling, or otherwise, into a slender configuration, so that it may be delivered through a narrow lumen of a laparascopic device. Flexibility of the implant is influenced by many factors, including, the materials from which the implant is made, treatments applied to the implant or any other features of the body of the implant.

A mesh implant may either include a single mesh or be formed from two or more mesh segments that are joined or overlap. In some embodiments, meshes are configured to continuously deliver at least one fibrous tissue inducing protein and optionally at least one tissue adhesive at the site of a fascia defect in a subject, thereby resulting in repair of the defect. It is contemplated that meshes can be configured to deliver at least one fibrous tissue inducing protein continuously, for example, for approximately 15 days, 20 days and 30 days. The length of time, however, will vary depending on the extent and site of the defect to repair, age of the patient and other clinical parameters that are typically taken into consideration by surgeons.

Surgical implants for use in the methods of the invention may be manufactured, sterilized, and contained in packages until opened for use in a surgical procedure. Any appropriate sterilization process can be used, including the conventional physical or chemical methods or treatment with ionizing radiation such as, for example, gamma or beta rays.

### Delivery Methods

Surgical implants and compositions described herein can be used in any of the surgical procedures that are used by surgeons for repair of a fascia tissue defect. In some embodiments, an incision is made at the site of a hernia in a subject and a surgical implant described herein is inserted to cover the area of the defect. In other embodiments, a laparoscopic method is used to deploy a surgical implant in the patient. Fascia tissue defect repair may be performed using general, regional, or local anesthesia. Some of the advantages of local anesthesia include a short recovery time and ability to test the repair intra-operatively. Further, local anesthesia avoids the respiratory and immune depressive effects of general anesthesia.

As previously noted, compositions described herein may be applied directly to the site of a fascia defect, injected at the site of the defect, or applied to a surgical implant before or after it is placed at the site of the fascia defect. In some embodiments, compositions described herein may be used in conjunction with a mesh which covers a fascia defect in a structure which normally contains an organ or a tissue, such as, for example, the abdominal wall. For example, compositions comprising a fibrous tissue inducing protein and optionally a tissue adhesive may be delivered to the site of a hernia using a device, suitable for administering the composition to or near the site of the hernia. Such a method of delivery would eliminate the need to treat or soak a mesh, or other surgical implant, in the composition prior to its implantation in a subject.

Various methods of hernia repair and implants suitable for use in hernia repair are known and described, for example, in U.S. Pat. Nos. 5,176,692; 5,569,273; 6,800,0825,824,082; 6,166,286; 5,290,217; and 5,356,432. Generally, such devices include (a) a mesh-like member configured for repairing a fascia defect in a subject; and optionally (b) a means for securing the mesh-like member to the site of the fascia. The devices of the invention are distinct in that the surgical implant or mesh-like member contains a therapeutically effective amount of one or more fibrous tissue inducing proteins and optionally, one or more tissue adhesives.

### Uses

Compositions and surgical implants described herein may be tested in a wide variety of well known and available animal models for repair of fascia tissue defects. For example, the strength of hernia repair can be tested according to porcine groin hernia repair stress-loading tests taught in Uen, “Comparative Laparoscopic Evaluation of the PROLENE polypropylene hernia system vs. the PerFix plug repair in a porcine groin hernia repair model,” J. Laparoendosc. Adv. Surg. Tech. 14(6):368-73 (2004). Light microscopy can also be used to evaluate the health of other structures near a hernia, as taught in Berndsen et al., “Does mesh implantation affect the spermatic cord structures after inguinal hernia surgery? An experimental study in rats,” Eur. Surg. Res. 36(5):318-22 (2004).

Is contemplated that in addition to repairing hernias and stimulating growth of fascia tissue, the methods of the invention may also be applied to repairing damage to fascia tissue associated with, for example, colon surgery, rectal surgery, plastic surgery, trauma, surgery, vascular surgery, pelvic floor repair, or a wound, as well as fascia defects caused by chronic strain and immobility.

Accordingly, this invention may be used to treat various types of fascia defects, including for example, serious hernias, recurrent hernias, hernias in patients with diabetes or other conditions that are associated with impaired wound healing, or any other fascia defects in patients with diabetes or other conditions that are associated with impaired wound healing.

The following examples are illustrative of the present invention and are not limiting in any manner. Modifications, variations and minor enhancements are contemplated and are within the scope of the present invention.

### EXAMPLES

The following materials and methods were used in the subsequent Examples. It will be appreciated by those of skill in the art that while the Examples employ BMP-12, they can be performed in a similar manner with BMP-13, MP-52, or any another fibrous tissue inducing protein. Similarly, other tissue adhesives and surgical implants may be substituted for those described in the Examples.

Various meshes employed as surgical implants in the following Examples include the Bard mesh which is a polypropylene mesh (PPM) and the Bard Composix mesh, which has two layers of PPM and a layer of expanded polytetraflouroethylene to minimize tissue adhesion to the mesh (Davol, Inc., Cranston, R.I.).

Additionally, the sepramesh biosurgical composite (Genzyme Surgical Products, Cambridge, Mass.) is also used, which includes PPM coated with chemically modified sodium hyaluronate/carboxymethylcellulose (HA/CMC). Examples of bioabsorbable meshes that may be used in the surgical implants described herein include the polyglactin vicryl mesh (Ethicon, Somerville, N.J.).

Various bioabsorbable sponges that may be employed as surgical implants include collagen sponges HELISTAT®, HELITENE® and VITAGUARD® (Integra Life Sciences, Plainsboro, N.J.), and ULTRAFOAM® (Davol, Inc., Cranston, R.I.).

Finally, the tissue adhesive TISSEEL® (Baxter Healthcare Corp., Deerfield, Ill.) is used to prepare a composition comprising TISSEEL® and rhBMP-12.

A. Preparation of Surgical Implants for Use in Hernia Repair

It is understood that any of the meshes and/or sponges that are currently available can be used as surgical implants. In the case of a mesh, the mesh is either coated with a composition including at least one fibrous tissue inducing protein, e.g., rhBMP-12, or it is impregnated with a composition comprising at least one fibrous tissue inducing protein.

Each of Bard mesh, Bard composix mesh, sepramesh biosurgical composite and the polyglactin vicryl mesh, following receipt from the manufacturer, are coated with a composition including rhBMP-12. Either both surfaces of the mesh or only one surface may be coated, such as the surface that faces outward from the defect after implantation, i.e., the dorsal surface. Additionally, the meshes are coated with an antibiotic to prevent infections in the area where the meshes are implanted. A suitable antibiotic can either be included in the same composition as the fibrous tissue inducing protein or it can be coated separately onto the mesh.

In some instances, the meshes are impregnated with a composition including a fibrous tissue inducing protein, e.g., rhBMP-12. This is achieved by cross-linking the fibrous tissue inducing protein to the fibers of the mesh before the fibers are interwoven into a mesh. However, it is expected that there will be no difference in hernia repair whether the meshes are coated or impregnated with a fibrous tissue inducing protein.

The sponges used in the surgical implant are either soaked in a composition including at least one fibrous tissue inducing protein or at least one fibrous tissue inducing protein can be cross-linked to the sponge material, for example, collagen. Cross-linking may be achieved using any suitable cross-linking agent.

B. Generation of an Animal Model for Hernia

An animal model for hernia is generated as follows. The guidelines for the animal study are in accordance with the NIH guidelines described in Guide for the Care of Laboratory Animals. (National Academy Press, 1996). Mature female New Zealand white rabbits (Oryctolagus cuniculuc), each weighing about 3.5-4.5 kg, are preanesthetized with acepromazine (0.5 mg/kg, sc). Ten to thirty minutes after administering the preanesthetic, animals are anesthetized with ketamine hydrochloride (30 mg/kg, im) and xylazine hydrochloride (10 mg/kg, im). The animals are intubated and fully anesthetized with isoflurane (1.0-3.0%) and oxygen (1.5-2.0 liters/min) followed by administration of buprenorphine (0.02-0.05 mg/kg, sc) as an analgesic.

The abdomen of each animal is shaved and prepped with a povidone/iodine scrub and successive alcohol wipes. A 10 to 12 cm skin incision is made beginning approximately 2 cm caudal to the xyphoid process and a 5 to 7 cm full-thickness muscular peritoneal abdominal wall defect is created by excising a segment around the linea alba. If necessary, the arteries are clamped for hemostatsis. The cecum of the animals is externalized from the abdominal cavity and abraded with a sterile nylon surgeon's brush. The cecum is visually divided into four sections and each section is abraded with 15 strokes such that punctate bleeding develops. The cecum is subsequently returned to the abdominal cavity and the animals are ready for implantation of a surgical implant and fibrous tissue inducing protein composition.

C. Histology

To evaluate fascia defect repair, the entire tissue area surrounding the original defect from each of the animal groups is excised and fixed in 4% paraformaldehyde (Polysciences, Warrington, Pa.) in PBS. The tissue specimens are embedded in paraffin and 5 μm thick sections are cut and stained with hematoxylin and eosin, and subject to a blind analysis. Morphologic characterization of cellular responses and tissue ingrowth is noted for each of the meshes.

D. Tissue Integration Strength Assay

A tissue integration assay is used to assess the strength of the tissue following hernia repair using the various methods described herein. Strips about 2×5 cm are cut parallel to the transverse axis of an implant, which includes the implant, the tissue/implant interface as well as normal tissue following recovery of the animals. The tensile strength of each tissue sample is measured using a tensiometer using a load cell. The maximum load at which the tissue/implant interface fails for each sample is recorded.

### Example 1

### A Mesh Coated with a Composition Containing a Fibrous Tissue Inducing Protein Designed for More Effective and Stronger Hernia Repair in an Animal Model

Rabbits are prepared as described above and are divided into two groups for each of the meshes: Bard mesh; Bard composix mesh and the sepramesh biosurgical composite. In each case, one group is implanted with the mesh prehydrated in sterile saline the other group is implanted with the mesh coated with a composition including a fibrous tissue inducing protein, rhBMP-12, as described above.

In each case, the mesh is secured to the 5×7 cm defect margin created as described above, with 3-0 Prolene in a simple continuous pattern. The subcutaneous tissue is closed with absorbable suture in a continuous subcuticular pattern. The animals are exuberated and allowed to recover in an incubator. In each of the six groups (i.e., Bard mesh and Bard mesh+rhBMP; Bard composix mesh and Bard composix mesh+rhBMP-12; and sepramesh biosurgical composite and sepramesh biosurgical composite+rhBMP-12), some of the animals are euthanized at about 15 days, some of the animals are euthanized at about 20 days and others are euthanized at approximately one-month after the surgery to monitor the overall performance of the treated and untreated meshes over time. The repair of the abdominal defect in each group is evaluated using the histological protocol and the tissue integration strength assays described above.

In each of the groups, it is predicted that the hernia repair will be stronger in the animal group which is implanted with the mesh coated with rhBMP-12, relative to the animals which are implanted with the meshes alone.

### Example 2

### A Surgical Implant Treated with a Composition Containing a Fibrous Tissue Inducing Protein Designed for Faster Repair of Hernia in an Animal Model

Rabbits are prepared as discussed above, and are divided into two groups. One group of rabbits is implanted with a PPM mesh and the other group of rabbits is implanted with a bioabsorbable sponge. Specifically, in one group, a hernia defect is covered with a PPM mesh coated with a composition including a fibrous tissue inducing protein, e.g., rhBMP-12, or a hernia defect is covered with a PPM mesh treated with a sterile saline solution, as discussed above. In the second group or rabbits, a hernia defect is either covered using a collagen sponge immersed in a composition including a fibrous tissue inducing protein, e.g., rhBMP-12, or it is covered with the sponge immersed in a sterile saline solution.

In each group of animals, the surgical implant, i.e., PPM mesh or the bioabsorbable sponge is secured to the 5×7 cm defect margin created using the method described above. The animals are allowed to recover and some of the animals from each group are euthanized at approximately one-month to evaluate the hernia repair.

In each of the groups, it is predicted that the hernia repair will be faster in the animal group which is implanted with the mesh coated with rhBMP-12, relative to the animals which are implanted with the meshes alone.

### Example 3

### A Composition Comprising BMP-12 and a Tissue Adhesive Designed to be Effective Alone or when Coated on a Mesh

Rabbits are prepared as described above and are divided into three groups: mesh alone, composition comprising rhBMP-12 and TISSEEL®, and the composition comprising rhBMP-12 and TISSEEL® applied to the mesh.

In the groups with a mesh, the mesh is secured to the 5×7 cm defect margin created as described above, with 3-0 Prolene in a simple continuous pattern. The subcutaneous tissue is closed with absorbable suture in a continuous subcuticular pattern. The animals are exuberated and allowed to recover in an incubator.

In the group with a composition comprising rhBMP-12 and TISSEEL®, the hernia is repaired surgically as described above and the rhBMP-12 and TISSEEL® composition is injected at the site of the hernia. The subcutaneous tissue is closed with absorbable suture in a continuous subcuticular pattern. The animals are exuberated and allowed to recover in an incubator.

In each of the three groups, some of the animals are euthanized at about 15 days, some of the animals are euthanized at about 20 days and others are euthanized at approximately one-month after the surgery to monitor the overall performance of the treatments over time. The repair of the abdominal defect in each group is evaluated using the histological protocol and the tissue integration strength assays described above.

It is predicted that the hernia repair will be faster in the animal group which is implanted with the mesh coated with rhBMP-12 and TISSEEL®, followed by the composition containing rhBMP-12 and TISSEEL®, relative to the animals which are implanted with the mesh alone.

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by this specification. All publications, patents, and sequences cited are incorporated by reference in their entirety. To the extent the material incorporated by reference contradicts or is inconsistent with the present specification, the present specification will supercede any such material. The citation of any references herein is not an admission that such references are prior art.

Unless otherwise indicated, all numbers expressing quantities of ingredients, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained. Unless otherwise indicated, the term “at least” preceding a series of elements is to be understood to refer to every element in the series. Those skilled in the art will recognize, or be able to ascertain using no more than routine experimentation, many equivalents to the specific embodiments described herein. Such equivalents are intended to be encompassed by the following claims.

What is claimed is:

1. A method of treating a fascia defect in a mammal, the method comprising administering to a site of the fascia defect a composition comprising a therapeutically effective amount of a fibrous tissue inducing protein.

2. The method of claim 1, wherein the fibrous tissue inducing protein is (1) at least 70% identical to BMP-12, BMP-13, or MP-52, or (2) a fragment of (1) capable of inducing fibrous tissue.

3. The method of claim 1, wherein the fibrous tissue inducing protein is BMP-12.

4. The method of claim 1, wherein the fibrous tissue inducing protein is BMP-13.

5. The method of claim 1, wherein the fibrous tissue inducing protein is MP-52.

6. The method of claim 1, wherein the fascia defect is associated with a wound.

7. The method of claim 1, wherein the fascia defect is associated with a hernia.

8. The method of claim 7, wherein the hernia is inguinal or femoral.

9. The method of claim 1, wherein the mammal is human.

10. The method of claim 1, wherein the mammal has diabetes.

11. The method of claim 1, wherein the composition further comprises a tissue adhesive.

12. The method of claim 11, wherein the tissue adhesive is selected from the group consisting of fibrin, fibrinogen, thrombin, aprotinin, Factor VIII, and 2-octyl cyanoacrylate.

13. The method claim 1, wherein the composition is delivered using a surgical implant configured for hernia repair.

14. The method of claim 13, wherein the surgical implant comprises a mesh.

15. The method of claim 14, wherein the mesh comprises polypropylene, polytetrafluoroethylene, polyurethane, or polyester.

16. The method of claim 14, wherein the mesh comprises a bioabsorbable material.

17. The method of claim 16, wherein the bioabsorbable material is collagen, gelatin, keratin, laminin, fibrin, fibronectin, alginate, hyaluronic acid, polyglycolic acid, polylactic acid, polyglycolide, or combination thereof.

18. The method of claim 13, wherein the surgical implant comprises an anti-adhesion compound or an adhesion barrier.

19. The method of claim 18, wherein the anti-adhesion compound is chemically modified sodium hyaluronate and carboxymethylcellulose, or hyaluronic acid, or collagen.

20. A hernia repair device, comprising: (a) a mesh-like member configured for repairing a fascia defect in a subject and comprising a therapeutically effective amount of a fibrous tissue inducing protein; and optionally (b) a means for securing the mesh-like member to the site of the fascia.

21. The device of claim 20, wherein the fibrous tissue inducing protein is (1) at least 70% identical to BMP-12, BMP-13, or MP-52, or (2) a fragment of (1) capable of inducing fibrous tissue.
